(19) United States
(12) Patent Application Publication
(10) Pub. No.: US 2020/0023021 A1
(43) Pub. Date: Jan. 23, 2020

(54) WEIGHT LOSS COMPOSITION INCLUDING CHLOROGENIC ACIDS AND PROBIOTICS

(71) Applicant: The Clorox Company, Oakland, CA (US)

(72) Inventors: Zachery T. Lewis, Pleasanton, CA (US); William Robert King, Pleasanton, CA (US); Francis C. Lau, Pleasanton, CA (US)

(21) Appl. No.: 16/511,157

(22) Filed: Jul. 15, 2019

Related U.S. Application Data
(60) Provisional application No. 62/700,531, filed on Jul. 19, 2018.

(51) Int. Cl. A61K 35/745; A61K 31/7034; A61K 35/747; A61K 9/48

(52) U.S. Cl. CPC A61K 35/745; A61K 31/7034; A61K 35/747; A61K 2035/115; A61K 9/4875; A61K 9/4816; A61K 9/4866

(57) ABSTRACT

Weight loss compositions including the combination of chlorogenic acids and probiotics are disclosed herein. The compositions aid weight loss by changing the mass balance equation of glucose in the gut from absorption through the intestinal wall and integration into adipose tissue to metabolism by the probiotic bacteria in the lumen. The growth of the probiotics is further boosted by incorporation of the chlorogenic acids.

### CROSS-REFERENCE TO RELATED APPLICATION(S)

The present disclosure claims priority to U.S. Provisional Application Ser. No. 62/700,531, filed Jul. 19, 2018, which is hereby incorporated by reference in its entirety.

### BACKGROUND OF THE DISCLOSURE

The present disclosure relates generally to weight loss compositions including the combination of chlorogenic acids and select probiotics. The composition aids weight loss by changing the mass balance equation of glucose in the gut from absorption through the intestinal wall and integration into adipose tissue to metabolism by the probiotic bacteria in the lumen. Particularly, the chlorogenic acids prevent absorption of glucose and alter glucose metabolism by the liver, and the glucose-metabolizing probiotic actively imports the sugar and breaks it down intracellularly. Further, it has been found that the growth of specific probiotics can be boosted by the presence of the chlorogenic acids, leading to synergy in suitable embodiments. It is particularly suitable for the compositions to be formulated having water activity (a_{w}) levels such as to support long-term viability of the probiotic, thus providing improved shelf-life stability.

The causes of the obesity crisis over the last 30 years remain unclear but are thought to relate primarily to increased consumption of energy-dense foods and beverages and decreased physical activity. In industrialized countries, affluence provides abundant and variable food items to the general public. Food, with the associated taste and olfactory pleasures, is an indulgence, not just for basic survival. As a result, obesity and obesity-related health issues (e.g., diabetes, hypertension, etc.) are increasing rapidly, and there is a strong need for dietary supplements that help with weight control. The market size for food supplements that decrease body weight is large, and there are few products that are both safe and effective.

Glucose metabolism plays important roles in the development of diabetes and obesity, and restricted uptake of glucose and its oligo- and polysaccharide precursors is an effective therapeutic means for diabetes and obesity. A substantial portion of glucose uptake in daily life comes from starch. After ingestion, starches are first broken down into complex sugars by amylase in saliva and in intestine. The complex sugars are then turned into glucose by glucosidases. Another major source of glucose uptake is from sucrose consumed every day. Sucrose, also called cane sugar, beat sugar, maple sugar and even “table sugar”, appears in most of the soft drinks and in all sorts of foods such as deserts. Sucrose is a disaccharide, consisting of one unit of glucose and one unit of fructose. After ingestion, sucrose is hydrolyzed into glucose and fructose by glucosidase in the small intestine. Finally, the glucose crosses the lining of the intestine, mainly through a sodium dependent glucose transporter and enters into the blood stream.

Existing therapies for obesity include standard diets and exercise, very low calorie diets, behavioral therapy, pharmacotherapy involving appetite suppressants, thermogenic drugs, food absorption inhibitors, mechanical devices such as jaw wiring, waist cords and balloons, and surgery.

Considering the high prevalence of obesity in society and the serious consequences associated therewith (e.g., adverse psychological development, dermatological disorders such as infections, varicose veins, exercise intolerance, diabetes mellitus, insulin resistance, hypertension, hypercholesterolemia, and coronary heart disease), any therapeutic composition potentially useful in reducing weight of obese persons could have a profound beneficial effect on their health. There is thus a need in the art for a composition that will reduce total body weight of obese subjects toward their ideal body weight without significant adverse side effects and that will help the obese subject maintain the reduced weight level. It would be further advantageous (especially in support of maintenance of normal blood sugar levels) if the composition could simultaneously provide for reduced glucose absorption by the intestine and increased metabolism of glucose by the gut microbiome.

### BRIEF DESCRIPTION

The present disclosure is directed to a weight loss composition that is capable of aiding weight loss by changing the mass balance equation of glucose in the gut from absorption through the intestinal wall and integration into adipose tissue to metabolism by the probiotic bacteria in the lumen. Generally, the weight loss composition includes at least one chlorogenic acid to reduce glucose absorption and at least one probiotic to increase non-human glucose metabolism in the gut.

In one aspect, the present disclosure is directed to a weight loss composition consisting essentially of at least one chlorogenic acid and at least one probiotic. The probiotic is selected from Bifidobacterium lactis (B. lactis), Lactobacillus gasseri (L. gasseri), Lactobacillus plantarum (L. plantarum) Lactobacillus acidophilus (L. acidophilus), Lactobacillus paracasei (L. paracasei), Lactobacillus rhamnosus (L. rhamnosus), Lactobacillus casei (L. casei), Streptococcus thermophiles (S. thermophiles), Bifidobacterium bifidum (B. bifidum), Bifidobacterium longum (B. longum) and combinations thereof.

In another aspect, the present disclosure is directed to a weight loss composition comprising at least one chlorogenic acid and at least one probiotic selected from the group consisting of Bifidobacterium lactis B420 (B. lactis B420), B. lactis BPL1, B. lactis BB-12, Lactobacillus gasseri BNR17 (L. gasseri BNR17), L. gasseri SBT2055, Lactobacillus casei DN001 (L. casei DN001), Lactobacillus acidophilus La-5 Bifidobacterium breve B-3 (B. breve B-3), a Lactobacillus plantarum (L. plantarum) mix (L. plantarum CECT 7527, L. plantarum CECT 7528, and L. plantarum CECT 7529) and combinations thereof.

In another aspect, the present disclosure is directed to a weight loss composition consisting essentially of at least one chlorogenic acid, at least one probiotic, and at least one prebiotic.

In yet another aspect, the present disclosure is directed to kits comprising a container and the above described weight loss compositions.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 depicts the mechanism of action of the weight loss composition of the present disclosure.

FIG. 2 depicts the effect of a commercially available cholorgenic acid powder (Svetol, Naturex—NJ, USA) on the growth of various bifidobacterial strains as analyzed in Example 1.

FIG. 3 depicts the positive effect of svetol on the growth of the Lactobacillus acidophilus La-14 strain (P<0.001) as analyzed in Example 2. Lactobacillus strains were grown in MRS used as the base medium, half of which was supplemented with an 18 g Svetol/L stock solution Svetol stock solution (MRS-S). Biolog Redox Dye G was aliquoted each MRS and MRS-S to a concentration of 2%. Two-hundred microliters of the control broth (MRS) was pipetted into each well of 96-well plates, with two replicate plates per strain of Lactobacillus and a background negative control plate. The same was done for the MRS-S broth for a total of 14 plates. Each culture was vortexed for 30 seconds, then serially diluted to 10-5 in 1% peptone water. On each plate, 10 μl of the diluted culture was placed onto the 96-well plate; this was repeated for the four plates for each strain. Wells were mixed by aspirating and dispensing the contents of each well repeatedly. Plates were incubated in the Omnilog Device (Biolog) for 24 hours at 36±1° C. The growth of two lactobacilli strains not shown (Lactobacillus gasseri Lg-36 and Lactobacillus plantarum Lp-115) were found to not be significantly affected by the presence of chlorogenic acid solutions. N=64 for each Lactobacillus strain, P values from two-sample T-tests.

FIG. 4 depicts the growth of several probiotic species. In this Example, the ability of Bifidobacterium spp. and Lactobacillus spp. to use Svetol as a sole carbon source was analyzed as in Example 3. A modified MRS (mMRS) broth was prepared without a carbon source by removing glucose from the preparation. The mMRS broth was used as the base medium, half of which was supplemented with the Svetol (MRS-S) to achieve a chlorogenic acid concentration of 50 μM (Laffay et al. 2006). The broth was heat sterilized at 116° C. for 15 minutes. Lactobacillus (LA14, LP299V) and Bifidobacterium (BL04, B420, Bi26) overnight cultures were prepared by inoculating MRS broth (BD Difco) or MRS Broth supplemented with 10 g galactose/L (MRS-gal) with a single colony from streak plates. The cultures were incubated for 18±2 hours at 36±1° C. under anaerobic conditions (AS-580, 90% N, 5% CO₂, 5% H). For each strain, the growth assay was performed by transferring 50 μl of overnight culture into three 15 ml falcon tubes containing 10 ml of mMRS-S and three tubes containing 10 ml of mMRS broth without chlorogenic acid. A negative control (no inoculum) for each media type (with or without Svetol) was also prepared. Tubes were incubated for 16±2 hours at 36±1° C. under anaerobic conditions. After incubation, the cultures were vortexed for 10 seconds and diluted 1:2 in distilled water. Absorbance (OD600) was immediately measured after dilution using a spectrophotometer (Beckmann DU530). The negative controls of each matching media type were used as the baseline to blank the meter. Statistical analysis was performed in MiniTab 18, using two-sample T-tests to determine significance. (P value <0.05 for significance, N=3 for each Bifidobacterium and Lactobacillus strain.

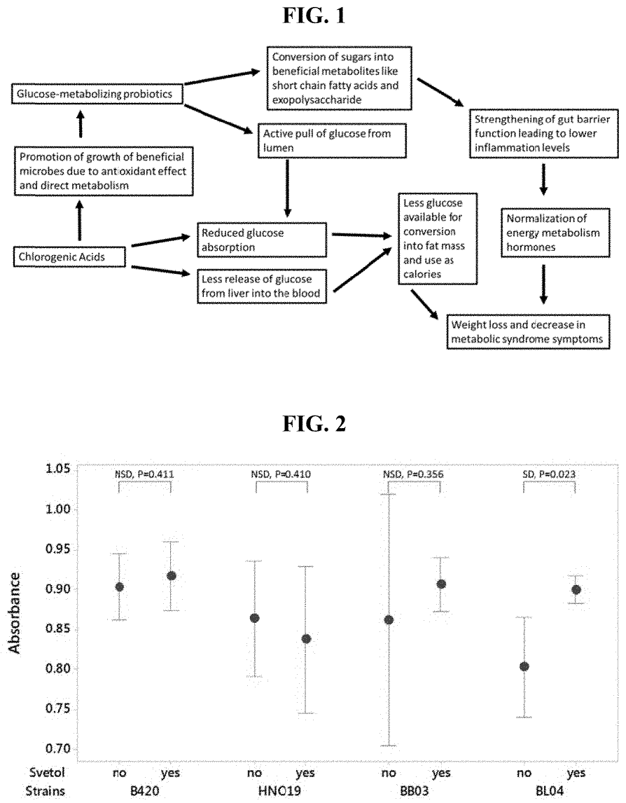

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

As used herein, the term “consisting of” is a closed term, limiting the composition to only the components listed after the term. “Consisting essentially of” refers to a composition including only the active components listed after the term and any additional non-active components, including, for example, diluents, excipients, preservatives, stabilizers, and the like, and combinations thereof.

Generally, the present disclosure is directed to a weight loss composition including at least one chlorogenic acid and at least one probiotic. In one suitable embodiment, the weight loss composition is in capsule form having improved stability and integrity due to the appropriate drying of raw materials and desiccation within its packaging. The composition aids weight loss as the chlorogenic acid prevents absorption of glucose and alters glucose metabolism by the liver. Further, the probiotic is a glucose-metabolizing probiotic that actively imports sugar (e.g., glucose, as well as other sugars) and breaks it down intracellularly.

Moreover, it is believed that by stimulating the availability of glucose in the small intestine, probiotic strains such as Bifidobacteria spp could experience a shorter lag time and have a greater success in colonizing the ileum and colon. This could be argued for several reasons: a) improved availability of an ideal substrate in the small intestine so that recovery from dormancy can begin sooner (i.e., reduced lag time), b) there may be less competition for carbohydrates in the small intestine versus the large intestine, where active colonic fermentation by competing groups such as enterobacteriaciae and mixed acid fermenters are already underway, and c) when probiotics reach the ileum in an already active state, they have a better chance to successfully colonize and to positively influence the pathways for fat metabolism by the liver, which is known to be impacted by the ileal microbiome. Faster activation and stimulation of Bifidobacteria spp should improve colonization in the large intestine, prospectively improving production of bifidobacterial metabolites that are known to positively impact fatty acid synthesis and deposition, i.e., short chain fatty acids. Further, greater Bifidobacteria spp growth can improve its ability to stimulate commensals like Akkermansia that are associated with reduced risk of obesity and metabolic syndrome.

Chlorogenic acids (CGA) are phenolic compounds formed by the esterification of cinnamic acids, such as caffeic, ferulic, and p-coumaric acids, with (−)-quinic acid. A series of health benefits have been associated with the consumption of CGA in the last few years, such as reduction of the relative risk of cardiovascular disease, diabetes type 2, and Alzheimer's disease, and antibacterial and anti-inflammatory activities. Their lactones also have been shown to exert positive effects in rats such as enhancement of insulin action.

Polyphenols as a class of compounds can be antibacterial and are used to decrease levels of undesirable bacteria. However, chlorogenic acids have been also shown in the past to affect the gut microbial community in potentially beneficial ways [Couteau, D., et al. (2001). Isolation and characterization of human colonic bacteria able to hydrolyse chlorogenic acid. Journal of Applied Microbiology, 90(6), 873-881; Mills, C. E., et al. (2015). In vitro colonic metabolism of coffee and chlorogenic acid results in selective changes in human faecal microbiota growth. British Journal of Nutrition, 113(8), 1220-1227.]. Accordingly, the compositions of the present disclosure include one or more of a number of commercially available probiotic bacteria whose growth is boosted by the presence of chlorogenic acids in a carbohydrate-source independent manner. This should contribute to the weight loss efficacy of the disclosed composition by increasing the sugar consumption via higher levels of probiotics. Thus, the overall effect of the compound on probiotics is likely strain-specific and care must be taken to pair them in a compatible manner.

Green (or raw) coffee is a major source of CGA in nature. Recent studies demonstrated that the consumption of green coffee extracts produced antihypertensive effects in rats and humans, improvement in human vasoreactivity, inhibitory effects on fat accumulation and body weight in mice and humans, and modulation of glucose metabolism in humans. The major CGAs in green coffee are 3-, 4- and 5-caffeoylquinic acid (3-, 4-, and 5-CQA), 3,4-, 3,5-, and 4,5-dicaffeoylquinic acid (3,4-, 3,5-, and 4,5-diCQA), 3-, 4-, and 5-feruloylquinic acids (3-, 4-, and 5-FQA), and 3-, and 4-, and 5-p-coumaroylquinic acids (3-, 4-, and 5-p-CoQA). Caffeoylferuloylquinic acids are minor CGA compounds also found in green coffee. Very small amounts of CGA lactones formed by heating during primary processing may also be observed.

In some suitable embodiments, the chlorogenic acid included in the compositions of the present disclosure includes from about 60% to about 75% by weight caffeoylquinic acid (CQA), more suitably, from about 65% to about 72% by weight caffeoylquinic acid, and even more suitably about 71% by weight caffeoylquinic acid.

In some embodiments, the caffeoylquinic acid providing the chlorogenic acid of the compositions is in the form of from about 20% to about 25% by weight 3-caffeoylquinic acid (3-CQA), suitably from about 20% to about 25% by weight 4-caffeoylquinic acid (4-CQA), and even more suitably from about 25% to about 30% by weight 5-caffeoylquinic acid (5-CQA).

In some suitable embodiments, the chlorogenic acid included in the composition includes from about 5% to about 15% by weight dicaffeoylquinic acid (diCQA), more suitably, from about 7% to about 10% by weight dicaffeoylquinic acid, and even more suitably about 9% by weight dicaffeoylquinic acid.

In some suitable embodiments, the chlorogenic acid included in the composition includes from about 5% to about 15% by weight feruloylquinic acid (FQA), more suitably, from about 10% to about 14% by weight feruloylquinic acid, and even more suitably about 13% by weight feruloylquinic acid.

In yet one particularly suitable embodiment, the chlorogenic acid includes from about 60% to about 75% by weight caffeoylquinic acid (CQA), from about 5% to about 15% by weight dicaffeoylquinic acid (diCQA), and from about 10% to about 20% by weight feruloylquinic acid (FQA).

In some embodiments, the chlorogenic acid included in the weight loss composition of the present disclosure further includes caffeoylferuloylquinic acid.

One particularly suitable source of chlorogenic acid includes green coffee bean extract, such as commercially available SVETOL® (Naturex, France).

Typically, the weight loss composition provides a dosage of from about 200 mg to about 225 mg chlorogenic acid per day.

The weight loss compositions of the present disclosure additionally include a probiotic, and in particular, a glucose-metabolizing probiotic. Suitable probiotic strains include, for example, one or more strains from the genus Lactobacillus (e.g., Lactobacillus rhamnosus, Lactobacillus acidophilus, Lactobacillus casei, Lactobacillus paracasei, Lactobacillus plantarum, Lactobacillus salivarius, Lactobacillus gasseri), one or more strains from the genus Bifidobacterium (e.g., Bifidobacterium animalis subsp. lactis, Bifidobacterium bifidum, Bifidobacterium longum, Bifidobacterium breve), one or more strains from the genera: Streptococcus (e.g., Streptococcus thermophiles), Lactococcus, Enterococcus, Leuconostoc, Akkermansia, Faecalibacterium and like probiotic strains that are sensitive to water activity. In one embodiment, the probiotic includes a combination of one or more strains of Lactobacillus and one or more strains of Bifidobacterium probiotic strain. In particular, the compositions include one or more probiotic strains selected from Bifidobacterium animalis subsp. lactis (B. lactis) (e.g., B. lactis B420, B. lactis BB-12 and/or B. lactis BPL1), Bifidobacterium breve (e.g., B. breve B-3), Lactobacillus gasseri (L. gasseri) (e.g., L. gasseri BNR17, L. gasseri SBT2055), Lactobacillus casei (e.g., L. casei DN001), Lactobacillus acidophilus (e.g., L. acidophilus La-5), Lactobacillus plantarum (e.g., L. plantarum CECT 7527, L. plantarum CEC 7528, L. plantarum CECT 7529), and combinations thereof. In suitable embodiments, confirmation of the ability of at least one probiotic in the composition to consume glucose should be confirmed either experimentally or genomically, as some probiotic species do not possess the ability to metabolize glucose. In especially suitable embodiments, the probiotic will have the above abilities to consume glucose in comparison to similar probiotic strains, as that would most effectively change the mass balance equation of glucose in the lumen.

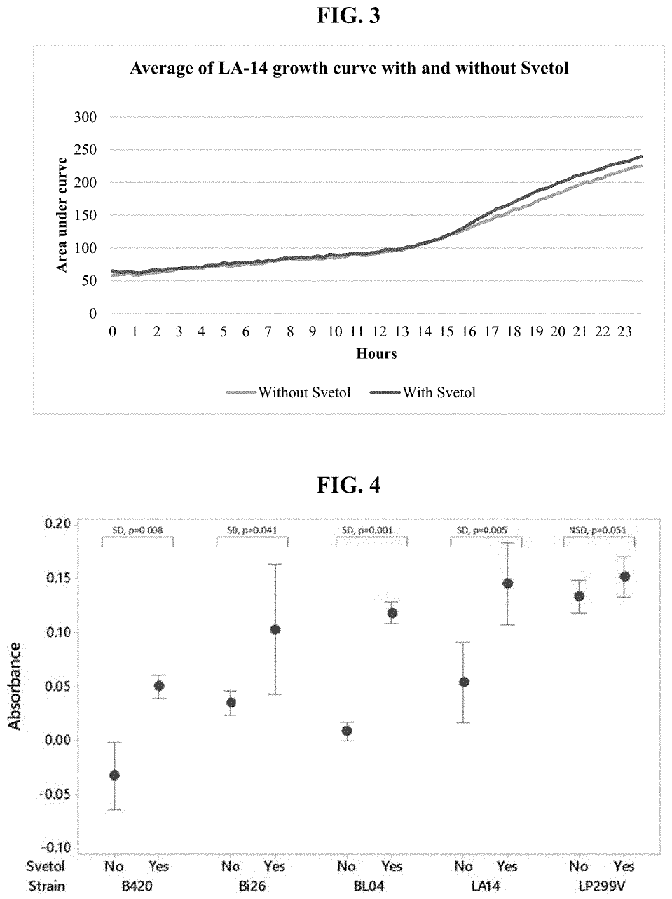

In suitable embodiments, confirmation of the ability of at least one probiotic in the composition to consume glucose should be confirmed either experimentally or genomically, as some probiotic species do not possess the ability to metabolize glucose. In especially suitable embodiments, the probiotic will have the ability to consume glucose to a greater degree than similar probiotic strains, as that would most effectively change the mass balance equation of glucose in the lumen. As disclosed herein, some probiotic strains show a boosted growth yield or growth rate in the presence of chlorogenic acids (FIGS. 2 & 3, respectively), and especially suitable embodiments will utilize strains exhibiting similar synergy such as Bl-04 and La-14 above. The synergy is likely achieved through some combination of the chlorogenic acid's antioxidant effect protecting the bacteria from oxidative damage and the bacteria possessing necessary enzymes (such as esterases) to benefit from hydrolysis of the compound. FIG. 4. demonstrates that only select probiotic species can utilize chlorogenic acids to grow in the absence of other carbohydrates sources, essentially showing a prebiotic effect of the chlorogenic acids. Not all probiotics will benefit from this synergy, as care should be taken to match the compound with probiotics with the right genomic abilities such as low intrinsic resistance to oxidative pressure, appropriate anaerobic/aerobic growth patterns, and ability to use the compound as a growth substrate. Indeed, some probiotics may be influenced more by the polyphenolic compound's antimicrobial properties of interacting with the cell wall and membrane than the desirable synergistic interactions. If the balance tips more towards probiotic growth promotion, regardless of the mechanism, the weight loss effects of the composition would be predicted to be greater due to greater elimination of available carbohydrates from the lumen and greater production of anti-inflammatory metabolites.

It has been found that the weight loss composition can be prepared to have a probiotic potency ranging from low potency (approximately 1 Billion CFU/serving) to higher potency (approximately 50 Billion CFU/serving). Typically, the weight loss composition has a potency ranging from about 10 Billion CFU/serving to about 30 Billion CFU/serving through the end of shelf life, including from about 10 Billion CFU/serving to about 20 Billion CFU/serving. It should be understood that the potency of the weight loss composition should be maintained at the desired ranges through end of shelf-life of the composition; that is, the potency of the weight loss compositions should remain within the range of greater than 1 Billion CFU/serving to 50 Billion CFU/serving through the end of shelf-life. As used herein, “shelf-life” refers to the period from the point of producing the finished product (i.e., weight loss composition), through packaging, shipping and handling, to storage of the packaged product, typically, for a storage period of up to 24 months, including a period ranging from about 12 months to about 24 months, and suitably from about 18 months to about 24 months. Typical storage temperatures range from about 0° C. to about 37° C., including about 4° C. to about 25° C.

In addition to the chlorogenic acid and probiotic strains, the weight loss compositions of the present disclosure may include at least one excipient. Typically, the weight loss composition may contain an excipient in addition to the chlorogenic acid and probiotics at a level of up to 95% or more by weight of the composition. Many functional excipients are agricultural in origin. Several of these excipient classes complement or enhance the functionality of probiotic cultures, in particular, selected oligosaccharides and fibers can boost the growth and performance of probiotic strains in the gastrointestinal (GI) tract after consumption. In addition, other excipients have been developed which enhance the immune support, regularity, or vaginal health effects of selected probiotic strains. Such excipients may be chosen to enhance and complement the functional benefits of a probiotic. Since probiotics are very sensitive to water activity (a_{w}) and are highly unstable in the presence of many excipients (including improperly treated Chlorogenic acids), many of which are also naturally hygroscopic (i.e., have a tendency to absorb moisture from the air), this results in undesirably high levels of a_{w}.

In some embodiments, the weight loss composition includes excipients that have the proven ability to support the growth of one or more of the probiotic strains used in the composition, such as prebiotic oligosaccharides, prebiotic fibers, and combinations thereof. Particularly, suitable excipients in these embodiments include xylo-oligosaccharide (XOS), fructo-oligosaccharide (FOS), galacto-oligosaccharides (GOS), inulin, aranbinoxylan, xylan, polydextrose (PDX), lactitol, pullulan, gentiobiose, and combinations thereof. Further, these prebiotics and fibers can be associated with microbiome effects like higher Akkermansia levels, higher levels of short chain fatty acids, improved Bifidobacterium levels, improved colonic fermentation, and improved overall weight and fat metabolism.

In other suitable embodiments, the excipients for use with the weight loss composition include, for example, dried fungal fermentates, yeasts, whole fruits, berries, botanicals, extracts, betaglucan, cereals, cellulose and the like, and combinations thereof.

In some suitable embodiments, the weight loss compositions are encapsulated into capsule form including a capsule made of a plant-derived water soluble polysaccharide. The capsule is formed using any encapsulation method as known in the art. Suitable plant-derived water soluble polysaccharides include hydrocolloids such as gums and starches derived from, for example, tapioca, acacia, locust bean, and the like, as well as combinations thereof. Gums and starches defined above may or may not be produced by fermentation or enzymatic modification of organic plant material to produce water binding hydrocolloids such as pullulan, xanthan, exopolysaccharides, and the like, and combinations thereof.

Other suitable forms for administering the weight loss compositions include use of the compositions as dietary supplements in the form of tablets, powders, liquid drinks, foods, and the like as known in the art.

As stated herein, the weight loss composition of the present disclosure must achieve a balance of water activity (a_{w}) (also referred to as water activation (a_{w})) of excipients (including chlorogenic acids) used therein that is low enough for probiotic stability. When used in the capsule form, it should further be appreciated that the components of the weight loss composition must be selected to achieve a water activity (a_{w}) high enough for capsule integrity. The weight loss compositions of the present disclosure are capable of achieving the ideal balance of water activity by using processes including control and treatment of raw ingredient water activity (a_{w}), selection of specific types of desiccant and desiccant levels for use with the chlorogenic acid, probiotics and excipients, selection of packaging types, and managing the internal equilibration of water activity between the capsule, probiotics, and excipients.

Water activity (a_{w}) represents the ratio of the partial water vapor pressure of a food to a partial water vapor pressure of pure water under the same conditions. Water activity is an important parameter in controlling water migration of multicomponent products. Undesirable changes are often the result of moisture migration in multicomponent foods and supplements. Moisture will migrate from the region of high a_{w} to the region of lower a_{w}, but the rate of migration depends on many factors such as, for example, relative hygroscopicity of the weight loss composition, unit dosage and desiccant. Hygroscopicity will be determined by the relative water binding capacity of the various ingredients. Water activity (a_{w}) of water is 1.0. Sample water activity can be determined using water activity equipment and measurement conditions as known in the art (e.g., Rotronic Water Activity Meter: HYGROLAB C1).

To measure water activity (a_{w}) of a probiotic or chlorogenic acid, for example, approximately 1.5 grams of probiotic powder is added to a sample container and covered until the measurement is taken. The sample container is then inserted into the sample holder or probe cavity after removing the lid of the sample container to take the water activity measurements. To measure water activity (a_{w}) of capsules, such as the embodiment of an encapsulated weight loss composition, empty capsules are placed in the sample container. There should be very little gap between each capsule as they are placed in a sample container. The number of capsules analyzed can vary based on capsule size. The sample container is then inserted into the sample holder or probe cavity to take the water activity measurements.

While control of initial a_{w} for the various components of the weight loss composition would seem to be readily attainable objectives, in reality it has proven difficult to work with specific chlorogenic acids and/or excipients without rapid absorption of moisture during the time required for blending and packaging, as well as during the storage and shelf life of finished compositions. Water activity (a_{w}) of excipients also varies on a lot-to-lot and supplier by supplier basis, depending on specific extraction and drying methods used as well as ambient conditions. The present disclosure describes additional tools that help to adjust and fine tune a_{w} levels from lot-to-lot in finished products.

The level of water activity that is needed for probiotic stability is ideally between 0.05 a_{w} and 0.15 a_{w} to ensure acceptable culture stability over time. Further, when in capsule form, capsules normally require higher water activities of 0.3-0.5 a_{w} in order to maintain sufficient tensile strength during encapsulation, bottling, shipping, and storage. Should the water activity drop below these levels, capsules become more brittle and begin to shatter at a high frequency. Thus, there is a gap between the ideal a_{w} for culture stability versus ideal a_{w} for capsule integrity. Narrowing this gap has been a critical component for enabling production of the encapsulated weight loss compositions of the present disclosure. As such, the encapsulated weight loss compositions of the present disclosure suitably have an initial water activity (a_{w}) at a temperature ranging from about 4° C. to about 37° C., including a temperature of about 4° C. to about 25° C., of less than 0.60, and more suitably, an initial water activity of from about 0.20 to less than 0.60, and more suitably, an initial water activity of from about 0.30 to less than 0.60, and even more suitably, less than 0.30.

Typically, the weight loss composition should include chlorogenic acid(s) having initial water activity (a_{w}) of less than 0.30, and suitably, from about 0.10 to about 0.25, to ensure that the resulting weight loss compositions have the desired water activity (a_{w}) at all time points from blending and packaging through storage and shelf life. Additionally, in embodiments of the present disclosure in which excipients are included in the weight loss composition, it should be understood that the excipients have an initial water activity (a_{w}) of less than 0.30, and suitably, from about 0.10 to about 0.20, to ensure that the resulting weight loss compositions have the desired water activity (a_{w}) at all time points.

To adjust and fine tune a_{w} levels, a process for combining these ingredients and maintaining stability is needed. Generally, a process for controlling a_{w} includes the steps of: calculating the amounts of chlorogenic acid, probiotics and excipients required for use in the weight loss compositions in accordance with desired dosages; blending the chlorogenic acid, probiotics and excipients to form a bulk composition with a desired initial a_{w}; if necessary, encapsulating the bulk weight loss composition and measuring a_{w}; filling a container (e.g., bottle) with the weight loss composition; adding an effective amount of desiccant to the container in accordance with the initial a_{w}; and equilibrating the contained product at a controlled rate by controlling temperatures, dessicant type and level, and package moisture vapor transfer rates (MVTR) to reach the desired a_{w}. The dessicant could be in the form of a pillow, canister or could be a dessicant layered bottle. Suitable dessicant types include, for example, silica gel, calcium oxide, molecular sieves, or a combination thereof.

Initially, the process requires selection and blending of ingredients to achieve the lowest possible starting a_{w}. Achieving an ideal a_{w} is not merely a matter of blending ingredients with low initial a_{w} levels. Virtually all suitable excipients of agricultural origin and of commercial value are very hygroscopic, which means that any exposure to humidity during production, storage, or blending results in rapid increases in water activity. Cultures are also highly hygroscopic and rapidly increase in a_{w} during handling. Accordingly, the probiotics and excipients for use in the encapsulated probiotic compositions are initially selected to include a desired initial a_{w} for each and in amounts that will provide the desired potency. Particularly, probiotic strains are selected alone or in combination to have an initial a_{w} of probiotics of less than 0.15 a_{w} and to provide a potency of at least 10 Billion CFU/serving. The chlorogenic acid and/or excipients are selected to have an initial a_{w} of less than 0.30 a_{w}, and suitably, from about 0.10 a_{w} to about 0.20 a_{w}, which can be achieved through the chilsonation process described below.

As noted above, however, selection of ingredients with desired initial a_{w} is not sufficient to ensure that the end composition can be prepared with a water activity to allow for stability. Accordingly, after determining the types and amounts of chlorogenic acid, probiotics and excipients to form the weight loss composition, chilsonation, a known mechanical milling process, has been adapted in the process of the present disclosure in order to improve blending and reduce initial ingredient water activities of the excipients to be used in the prepared weight loss compositions of the present disclosure. Normally, chilsonation is a milling treatment that can be used to adjust particle size and bulk density of powdered ingredients. Particularly, chilsonation is a process of dry agglomeration. This treatment was modified by use of specific settings and specific components (e.g., rotors, power settings, gap sizes, screw speeds) to adjust initial excipient water activities. That is, the present disclosure utilizes a range of chilsonation settings that allow for the reduction of water activities over initial levels by up to 25% to 50%, moving initial a_{w}'s into a much more favorable range for blending and packaging. Particularly, the chilsonation process is performed on the individual excipients as needed and is used to reduce the water activity (a_{w}) of the individual excipients from about 0.2 a_{w} to about 0.1 a_{w} prior to being blended with the chlorogenic acid and/or probiotics; that is, the chilsonation process has been adapted herein to be a drying process that does not damage active ingredients.

Additionally, even under the best possible blending and processing conditions, it is many times still not possible to ensure a final product water activity that meets product needs. For example, the water activity of filled capsules changes in significant and non-intuitive ways during the first month after packaging into bottles. Particularly, a low initial blended a_{w} gives way to higher a_{w} in bulk capsules as free moisture first begins to migrate from capsule to contents (the blend of chlorogenic acid, excipient and probiotic cultures), causing an initial increase in a_{w}. Next, a_{w} enters a phase of decline, as free moisture migrates from the capsules into the desiccant canisters or pillows that are present in the bottles, or in dessicant layered bottles or blister packs. After hitting a low point in ˜2 weeks at 5° C., the a_{w} again begins to rise due to additional migration and equilibration of water, until it reaches an equilibrium level around 4 weeks that will determine the overall longevity and stability of the active culture during shelf life. These changes show that the initial a_{w} is not completely predictive of where a composition will end up for long term a_{w}. The rate and extent of moisture migration during equilibration are key variables for creation of a stable end composition.

Typically, there is a tradeoff between water activity and breakage when encapsulated weight loss compositions (also referred to as capsules herein) have been conditioned at 25° C. for up to 4 weeks. By drying capsules to different a_{w} levels and then conducting a crush test, it is possible to determine the minimum breakage water activity threshold, defined as the a_{w} at which 50% of capsules will break during a crush test. Particularly, a weight (99.4 grams) is placed inside a hollow portion of a cylinder. This weight is held at the top by a lever. The capsule to be tested is placed on a flat surface and lever and weight assembly is placed above the capsule. Then the lever is released. This enables the weight to travel 4 inches before it hits the capsule. If the capsule is brittle, it causes a breakage. This type of breakage helps determine if the empty/filled capsule is brittle. This enables prediction of whether or not shipping and handling would cause breakage of capsules when shipped and stored under certain conditions.

By conducting crush testing on up to 30 capsules at each a_{w} point, a curve can be derived showing the exact breakage point. Temperature during the equilibration period has been determined to be a critical variable that determines the tendency of capsules to break, and can be manipulated as part of the equilibration process.

It has been discovered that the degree of capsule breakage can be manipulated by the rate at which moisture is removed from the capsules and the contents. One step involves tailoring the amount and form of desiccant to the overall moisture load in each end composition product (e.g., weight loss composition capsule).

By determining desired levels of desiccant and lowering the equilibration temperature, it becomes possible to generate stable capsules with lower a_{w} levels.

The present disclosure is further directed to kits including the weight loss compositions and containers for packaging the compositions. That is, once prepared, the weight loss compositions can be packaged into a container for sale to consumers. Suitable containers include bottles, canisters, blister packs, stick packs (form-fill-seal flexible packaging), and vials, and the like.

### EXAMPLES

### Example 1

In this Example, the effect of cholorgenic acid powder (Svetol, Naturex—NJ, USA) on the growth of various bifidobacterial strains was analyzed.

Bifidobacterium strains were assayed for growth in 15 ml falcon tubes with MRS-gal used as the base medium, half of which was supplemented with the Stevol stock solution (MRS-gal-S) to achieve a chlorogenic acid concentration of 50 μM, mimicking physiological conditions in the gut lumen. For each strain, 50 μl of overnight culture were used to inoculate each of six tubes; three containing five milliliters of MRS-gal and three containing MRS-gal-S. A negative control for each media type was also prepared. Tubes were incubated for 16±2 h at 36±1° C. under anaerobic conditions. After incubation, the cultures were vortexed for 10 seconds and diluted 1:2 in distilled water. Absorbance (OD600) was immediately measured after dilution using a spectrophotometer (Beckmann DU530). The negative controls were used as the baseline to blank the meter. N=3 for each Bifidobacterium strain, P values from two-sample T-tests.

### Example 2

In this Example, the positive effect of Svetol on the growth of the Lactobacillus acidophilus La-14 strain (P<0.001) was analyzed.

Lactobacillus strains were grown in MRS used as the base medium, half of which was supplemented with an 18 g Svetol/L stock solution Svetol stock solution (MRS-S). Biolog Redox Dye G was aliquoted each MRS and MRS-S to a concentration of 2%. Two-hundred microliters of the control broth (MRS) was pipetted into each well of 96-well plates, with two replicate plates per strain of Lactobacillus and a background negative control plate. The same was done for the MRS-S broth for a total of 14 plates. Each culture was vortexed for 30 seconds, then serially diluted to 10-5 in 1% peptone water. On each plate, 10 μl of the diluted culture was placed onto the 96 well plate; this was repeated for the four plates for each strain. Wells were mixed by aspirating and dispensing the contents of each well repeatedly. Plates were incubated in the Omnilog Device (Biolog) for 24 hours at 36±1° C. The growth of two lactobacilli strains not shown (Lactobacillus gasseri Lg-36 and Lactobacillus plantarum Lp-115) were found to not be significantly affected by the presence of chlorogenic acid solutions. N=64 for each Lactobacillus strain, P values from two-sample T-tests.

### Example 3

In this example, the ability of Bifidobacterium spp. and Lactobacillus spp. to use Svetol as a sole carbon source was analyzed

A modified MRS (mMRS) broth was prepared without a carbon source by removing glucose from the preparation. The mMRS broth was used as the base medium, half of which was supplemented with the Svetol (MRS-S) to achieve a chlorogenic acid concentration of 50 μM. The broth was heat sterilized at 116° C. for 15 minutes. Lactobacillus (LA14, LP299V) and Bifidobacterium (BL04, B420, Bi26) overnight cultures were prepared by inoculating MRS broth (BD Difco) or MRS Broth supplemented with 10 g galactose/L (MRS-gal) with a single colony from streak plates. The cultures were incubated for 18±2 hours at 36±1° C. under anaerobic conditions (AS-580, 90% N, 5% CO₂, 5% H).

For each strain, the growth assay was performed by transferring 100 μl of overnight culture into three 15 ml falcon tubes containing 10 ml of mMRS-S and three tubes containing 10 ml of mMRS broth without chlorogenic acid. A negative control for each media type was also prepared. Tubes were incubated for 16±2 hours at 36±1° C. under anaerobic conditions. After incubation, the cultures were vortexed for 10 seconds. Absorbance (OD₆₀₀) was immediately measured after dilution using a spectrophotometer (Beckmann DU530). The negative controls of each matching media type were used as the baseline to blank the meter.

Statistical analysis was performed in MiniTab 18, using two-sample T-tests to determine significance. (P value <0.05 for significance, N=3 for each Bifidobacterium and Lactobacillus strain.)

### Example 4

Exemplary Encapsulated Formulation

| | Total probiotic | | Input Qty | |
|---|---|---|---|---|
| | CFUs at end of | | per capsule | |
| Ingredients | shelf life | | (mg) for | |
| (Daily dose | (Billion CFUs | | non- | % of |
| is 2 capsules) | per capsule) | | probiotics | Formula |
| Lactobacillus | 5.00 | | | 5.87% |
| gasseri | | | | |
| BNR17 | | | | |
| Lactobacillus | 1.00 | | | 0.41% |
| plantarum | | | | |
| Lactobacillus | 0.75 | | | 0.86% |
| acidophilus | | | | |
| Lactobacillus | 0.38 | | | 0.17% |
| paracasei | | | | |
| Lactobacillus | 0.18 | | | 0.09% |
| rhamnosus | | | | |
| Lactobacillus | 0.20 | | | 0.17% |
| casei | | | | |
| Streptococcus | 0.25 | | | 0.09% |
| thermophilus | | | | |
| Bifidobacterium | 2.25 | | | 0.78% |
| lactis | | | | |
| Bifidobacterium | 0.002 | | | 0.09% |
| bifidum | | | | |
| Bifidobacterium | 0.001 | | | 0.09% |
| longum | | | | |
| Organic Svetol | | | 259.04 | 30.02% |
| (about 50% | | | | |
| chlorogenic acid) | | | | |
| Chilsonated | | | 381.85 | 44.25% |
| Organic Xylo- | | | | |
| oligosaccharide | | | | |
| Rice extract | | | 14.90 | 1.73% |
| blend | | | | |
| Rice fiber | | | 14.90 | 1.73% |
| Capsule | | Fill Wt.: | 745.00 | 86.33% |
| Vegetable | | Cap Wt.: | 118.00 | 13.67% |
| Pullulan | | | | |
| capsule, clear, | | | | |
| size “00” | | | | |
| | | Total wt.: | 863.00 | 100.00% |

The previous exemplary weight loss composition embodiment is provided solely by way of example and is not intended to limit the scope of the present disclosure in any way. Consistently, various other formulation embodiments of the weight loss compositions and methods of manufacture and packaging the same, within the scope of the present disclosure are disclosed herein.

### Example 5

Exemplary Non-Capsule Formulation

| | Total probiotic | |
|---|---|---|
| | CFUs at end of shelf | Grams per |
| | life (Billion | dose (for |
| Ingredients | CFUs/per dose) | non-probiotics) |
| Lactobacillus gasseri BNR17 | 10.00 | |
| Lactobacillus plantarum mix | 2.40 | |
| (CECT 7527, 7528, and 7529) | | |
| Lactobacillus acidophilus La-5 | 2.50 | |
| Bifidobacterium breve B3 | 5.00 | |
| Lactobacillus gasseri SBT2055 | 16.00 | |
| Lactobacillus casei DN001 | 2.00 | |
| Bifidobacterium lactis BB-12 | 2.50 | |
| Bifidobacterium lactis B420 | 10.00 | |
| Bifidobacterium lactis BPL1 | 10.00 | |
| Organic Svetol (about 50% | | 0.500 |
| chlorogenic acid) | | |
| Polydextrose | | 12.00 |

The previous exemplary weight loss composition embodiments are provided solely by way of example and are not intended to limit the scope of the present disclosure in any way. Consistently, various other formulation embodiments of the weight loss compositions and methods of manufacture and packaging the same, within the scope of the present disclosure are disclosed herein.

What is claimed is:

1. A weight loss composition consisting essentially of at least one chlorogenic acid and at least one probiotic selected from the group consisting of Bifidobacterium lactis (B. lactis), Lactobacillus gasseri (L. gasseri), Lactobacillus plantarum (L. plantarum) Lactobacillus acidophilus (L. acidophilus), Lactobacillus paracasei (L. paracasei), Lactobacillus rhamnosus (L. rhamnosus), Lactobacillus casei (L. casei), Streptococcus thermophiles (S. thermophiles), Bifidobacterium bifidum (B. bifidum), Bifidobacterium longum (B. longum) and combinations thereof.

2. The weight loss composition as set forth in claim 1, wherein the chlorogenic acid is selected from the group consisting of caffeoylquinic acid (CQA), dicaffeoylquinic acid (diCQA), feruloylquinic acid (FQA), p-coumaroylquinic acid (p-CoQA), and combinations thereof.

3. The weight loss composition as set forth in claim 2, wherein the chlorogenic acid further comprises caffeoylferuloylquinic acid.

4. The weight loss composition as set forth in claim 1, wherein the composition provides a dosage of from about 200 mg to about 225 mg chlorogenic acid per day.

5. The weight loss composition as set forth in claim 1 having a potency of at least 10 Billion CFU through the end of shelf-life.

6. The weight loss composition as set forth in claim 1 further comprising at least one excipient.

7. The weight loss composition as set forth in claim 6, wherein the at least one excipient is selected from the group consisting of prebiotic oligosaccharides, prebiotic fibers, and combinations thereof.

8. The weight loss composition as set forth in claim 7, wherein the at least one excipient is selected from the group consisting of xylo-oligosaccharide (XOS), fructo-oligosaccharide (FOS), Inulin (fiber), aranbinoxylan (fiber), polydextrose, and combinations thereof.

9. The weight loss composition as set forth in claim 6, wherein the at least one excipient is selected from dried fungal fermentates, yeasts, whole fruits, berries, botanicals, extracts, betaglucan, cereals, cellulose and combinations thereof.

10. The weight loss composition as set forth in claim 1, wherein the composition is encapsulated within a capsule comprising a plant-derived water soluble polysaccharide.

11. A weight loss composition comprising at least one chlorogenic acid and at least one probiotic selected from the group consisting of Bifidobacterium lactis B420 (B. lactis B420), B. lactis BPL1, B. lactis BB-12, Lactobacillus gasseri BNR17 (L. gasseri BNR17), L. gasseri SBT2055, Lactobacillus casei DN001 (L. casei DN001), Lactobacillus acidophilus La-5 Bifidobacterium breve B-3 (B. breve B-3), a Lactobacillus plantarum (L. plantarum) mix (L. plantarum CECT 7527, L. plantarum CECT 7528, and L. plantarum CECT 7529) and combinations thereof.

12. The weight loss composition as set forth in claim 11, wherein the chlorogenic acid is selected from the group consisting of caffeoylquinic acid (CQA), dicaffeoylquinic acid (diCQA), feruloylquinic acid (FQA), p-coumaroylquinic acid (p-CoQA), and combinations thereof.

13. The weight loss composition as set forth in claim 12, wherein the chlorogenic acid further comprises caffeoylferuloylquinic acid.

14. The weight loss composition as set forth in claim 11, wherein the composition provides a dosage of from about 200 mg to about 225 mg chlorogenic acid per day.

15. The weight loss composition as set forth in claim 11 having a potency of at least 10 Billion CFU through the end of shelf-life.

16. The weight loss composition as set forth in claim 11 further comprising at least one excipient.

17. The weight loss composition as set forth in claim 16, wherein the at least one excipient is selected from the group consisting of prebiotic oligosaccharides, prebiotic fibers, and combinations thereof.

18. The weight loss composition as set forth in claim 11, wherein the composition is encapsulated within a capsule comprising a plant-derived water soluble polysaccharide.

19. A weight loss composition consisting essentially of at least one chlorogenic acid, at least one probiotic, and at least one prebiotic.

20. A kit comprising a container and the weight loss composition of claim 1.
